(19) United States
(12) Patent Application Publication
(10) Pub. No.: US 2007/0010006 A1
(43) Pub. Date: Jan. 11, 2007

(54) System and method for detecting biohazardous threats

(75) Inventors: Douglas B. Quine, Bethel, CT (US); Ray Pan, Oxford, CT (US); Denis J. Stemmle, Stratford, CT (US)

Correspondence Address: PITNEY BOWES INC.;35 WATERVIEW DRIVE, P.O. BOX 3000, MSC 26-22, SHELTON, CT, 06484-8000, US

(73) Assignee: Pitney Bowes Incorporated, Stamford, CT (US)

(21) Appl. No.: 11/170,460

(22) Filed: Jun. 29, 2005

(51) Int. Cl. C12M 1/00

(52) U.S. Cl. 435/287.1

(57) ABSTRACT

A system and method for detecting a mailpiece contaminated by hazardous agents/materials including at least one contaminant sensor for detecting the presence of a hazardous agent/material and a Radio Frequency Identification (RFID) device in communication with the contaminant sensor. The contaminant sensor issues an alert signal to the RDIF device indicating that a hazardous agent/material has been sensed. The RFID device communicates a signal concerning the current status of the RFID device i.e., indicative of whether the RFID device and, consequently, the mailpiece has been in, or proximal to, the hazardous agent/material.

### FIELD OF THE INVENTION

The present invention relates generally to detecting biohazardous threats, more particularly, to a system and method of determining the presence of such biohazardous threats in mailpieces for delivery.

### BACKGROUND OF THE INVENTION

In late 2001, several United States postal offices and other buildings along the eastern United States were contaminated with Bacillus anthracis spores (anthrax), resulting in as many as twenty-two cases of anthrax infection. These incidents were costly in terms of the health-related impact and the required decontamination efforts. Cleanup following the anthrax contamination proved to be difficult, labor-intensive, and expensive. As this threat still exists, there is a need to detect biological contaminants within the postal packages or other containers. Similar attacks through the mail system are possible using other hazardous substances such as nerve or blistering agents, or any other substance which can harm any person who handles the contaminated mail piece.

Upon detecting a biohazardous threat on or in a mailpiece, it is desirable to trace the prior path of the mailpiece insofar as cross-contamination of other mailpieces can have serious adverse consequences. Exposure to a dose of anthrax invisible to the naked eye can produce serious health problems for those coming in contact with the deadly bacteria. In the past, tracing the history of a contaminated mailpiece has proven extremely difficult in view of the lack of adequate procedures or tracking systems internal to the mail distribution system, whether governmental or commercial.

A need, therefore, exists for an improved system and method for detecting, and/or tracking the origin/prior history of a mailpiece contaminated with a biohazardous agent/material.

### SUMMARY OF THE INVENTION

A system and method is provided for detecting a mailpiece contaminated by hazardous agents/materials including at least one contaminant sensor for detecting the presence of a hazardous agent/material and a Radio Frequency Identification (RFID) device also proximal to the mailpiece and in communication with the contaminant sensor. The contaminant sensor issues an alert signal to the RFID device that a hazardous agent/material has been detected. The RFID device communicates a signal concerning the current status of the RFID device i.e., indicative of whether the RFID device and, consequently, the mailpiece has been in or proximal to the hazardous agent/material.

### BRIEF DESCRIPTION OF THE DRAWINGS

Reference is now made to the various figures wherein like reference numerals designate similar items in the various figures and in which:

FIG. 1 is a schematic block diagram of a system for detecting hazardous agents/materials in or surrounding a mailpiece including a contaminant sensor for issuing an alert signal to a Radio Frequency Identification (RFID) device.

FIG. 2 is a schematic representation of a contaminant sensor for detecting hazardous agents/materials.

FIG. 3 is a schematic illustration of an exemplary RFID Device.

FIG. 4 is a table illustrating one embodiment of the invention wherein a checksum algorithm is used in combination with an RFID identification number to communicate a change in an RFID state.

FIG. 5 is a schematic diagram of a detection device comprising a contaminant senor integrated with or imbedded in an RFID device.

FIG. 6 is a schematic block diagram of another embodiment of the system wherein a computer processor tracks the history/prior location of contaminated mailpieces for determining the origin thereof or possible cross-contamination with other mailpieces.

FIG. 7 is a flow diagram of the method steps employed in the practice of the invention.

### DETAILED DESCRIPTION OF THE INVENTION

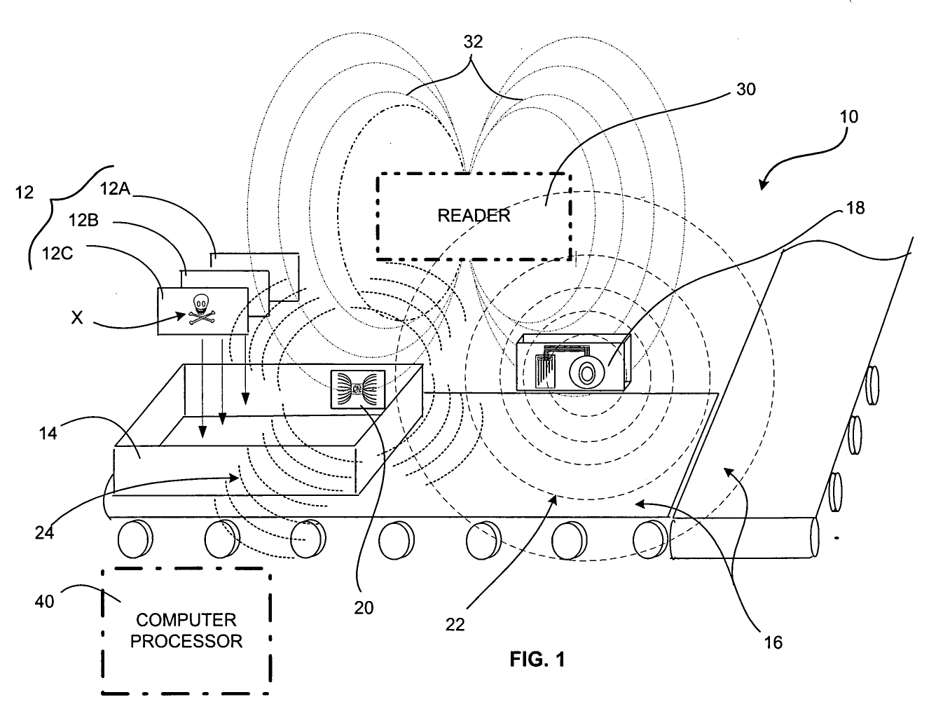

The present invention is described in the context of a mail distribution system which employs various trays or sorting equipment for bundling and/or routing mail; however, the teachings of the invention may be applied to individual mail pieces, i.e., whether or not trays or other sortation equipment are employed. In FIG. 1, a schematic is shown of a system 10 for detecting the presence of a contaminant on or within mailpieces 12. In the context used herein “contaminated” means any harmful or hazardous agent, material, toxin, pathogen, etc., whether organic or inorganic. The mailpieces 12A, 12B, 12C are contained within a mail tray 14 which is disposed on a transport deck 16 to illustrate movement of the mail pieces 12. In large mail distribution centers, it is common to have an extensive network of conveyors to transport mail within and about the facility. For the purposes of setting forth an example, a mailpiece 12C has been contaminated by a hazardous agent/material such as Ricin, Anthrax or Tularemia, etc., symbolically depicted by a generic symbol X for a dangerous/harmful substance.

In the broadest sense of the invention, the system 10 comprises a contaminant sensor 18 disposed proximal to the mailpiece 12C for detecting the presence of a hazardous agent/material, and an Radio Frequency Identification (RFID) device 20 proximal to and in communication with the contaminant sensor 18. In the context used herein, “proximal” means that the contaminant sensor 18 and RFID device 20 are sufficiently close to each other to enable electronic communication therebetween. This may include a wireless or a direct electrical connection including a configuration wherein the contaminant sensor is imbedded within or on the RFID device 20.

The contaminant sensor 18 is adapted to issue an alert signal 22 indicating that a hazardous agent/material has been sensed. In response to the alert signal 22, the RFID device 20 is adapted to change its status to provide notification that at least one of the mailpieces 12C is contaminated. More specifically, the RFID device 20 in combination with an RFID reader 30, (collectively referred to as the RFID system 20, 30) provides a change of status signal 24 for the purpose of providing a warning/advisory to facility operators.

In other embodiments of the invention, the RFID system 20, 30 communicates with a computer processor 40 to compile, store data and process information related to the location of the RFID devices 20 to augment the functionality of the system 10. These embodiments will be described in greater detail below.

A variety of contaminant sensors 18 may be used to detect threats and provide an alert signal to the RFID device 20 in accordance with the teachings of the invention. These include, inter alia, magnetoelastic, MicroElectroMechanical (MEM), microphysiometer, nanowire, waveguide, liquid crystal, distributed dust or DNA bridge sensors. A brief description of each is provided in subsequent paragraphs; however, more detailed information of each is readily available in the open literature.

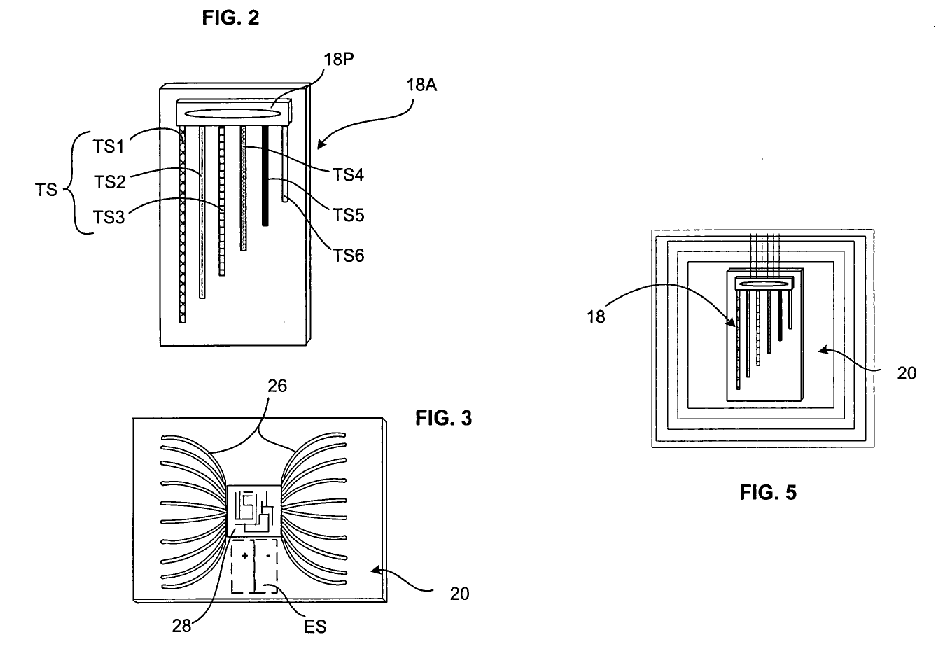

A Magnetoelastic sensor 18A (shown schematically in FIG. 2) monitors a change in resonance of a tuned magnetoelastic element/strip TS which has been coated with an antibody of the toxin to be detected. The antibodies on the surface of the magnetoelastic strip TS bond with the toxin (when present), thereby changing the mass, and consequently, the resonant frequency of the element. This change can be detected/measured to issue the alert signal 22. To detect multiple toxins, multiple individual strips TS1-TS6 or elements may be coated with respective antibodies. The strips TS1-TS6 may be ganged together and monitored by a common computer chip 18P containing the detection algorithms for issuing a unique alert signal for each detected toxin.

A MicroElectroMechanical (MEM) sensor (not shown) employs a similar teaching to that of Magnetoelastic sensors inasmuch as the MEM sensor also monitors changes in the resonance of a spring-mass system. In the case of MEM sensors, however, a small cantilever beam only four (4) microns in length and twenty (20) nanometers thick is employed to capture as little as a single anthrax spore or virus particle. Using a special adhesive, i.e., a selective antibody that adheres only to a certain type of toxin, a particle thereof attaches to the cantilever beam to effect a change in mass, and, consequently, the resonant frequency of the cantilever beam.

A Microphysiometer sensor (also not shown) employs live human cells that have been adapted to react quickly to biological agents in the environment. These cells are disposed atop sensors that rapidly detect abnormalities in cell structure. Hence, a cell that normally takes several days to break down in the presence of a toxin, e.g., Ricin, can react in only minutes, enabling advance detection. Cells of this type, i.e., those capable of rapid degradation, have been developed for nearly every vital organ such as the heart, liver, lungs and kidneys.

Nanowire or DNA bridge sensors (not shown) employ strings of DNA disposed in or completing an electrical circuit which changes conductivity or resistance as receptors in the DNA molecule accept or combine with other DNA molecules. These DNA strings can be adapted to receive or combine with harmful viral or bacterial DNA such as smallpox to detect and issue an alert signal.

Waveguide sensors (not shown) employ a coating of antibodies which are disposed on a sensor surface and selected to target specific bacteria cells. When the antibodies come into contact with these bacteria, the antibodies attack and destroy the bacteria and a light source is used to illuminate the changes. As the antibodies destroy the bacteria, the sensor surface detects the changes allowing the bacteria to be identified.

Liquid crystal sensors (not shown) employ cell membranes disposed atop rod-shaped liquid crystals to detect pathogens and other toxic agents. In this sensor, lipids are attached to the liquid crystals, which lay perpendicular to the surface and appear dark. When the sensor is exposed to an aqueous solution containing a protein that binds to the lipids, the liquid crystal molecules rapidly respond by switching to a planar orientation. As a result, the crystals transmit polarized light and appear bright. The change in illumination can be detected to issue an alert signal.

Distributed dust sensors (not shown) employ micrometer size particles which change color in the presence of contaminate More specifically, each particle exhibits different colors depending upon its orientation such that when attaching to a particular contaminate, the particles collectively yield a characteristic optical signature. The change in optical signature can be detected to issue an alert signal.

Immunoassay sensors (not shown) employ reactive materials which change color or contrast in the presence of a hazardous biological agent. The sensor includes a white absorptive stick coated with the reactive material which, upon contaminant exposure, effects a color change (i.e., becomes darker) against the white background. Sensors of this type are available from Osborn-Scientific located in Lakeside, Ariz., under the trade name BADD, an acronym for Biowarfare Agent Detection Device.

While several of the contaminant sensors described above do not provide an electrical output or switch closure, required for receipt by an RFID device, it will be appreciated that such sensors can be readily adapted to provide the necessary output. For example, changes in color, contrast or other physical characteristics can be converted to an electrical output/switch closure by conventional photoelectric or optical devices.

In FIG. 3, the RFID device 20 employed in the present invention can be active, passive or a hybrid thereof. A passive RFID device 20 includes an antenna 26 sufficiently large or selectively shaped/dimensioned to capture sufficient energy from a surrounding electromagnetic field to power the device 20. The antenna 26 is electrically connected to a central electronic chip 28 which performs the various preprogrammed RFID functions. An RFID reader 30 used in conjunction with passive RFID devices generates the necessary electromagnetic field 32 (shown in dashed lines in FIG. 1) of sufficient intensity or magnetic flux to power the device 20, i.e., when the device 20 is proximal to the reader 30. While this technology is continuously improving, readers 30 can produce a field such that a device 20 located therein can be energized and interrogated by the reader 30 at distances of up to ten (10) feet.

An active RFID device 20 includes an energy source ES, e.g., an embedded battery, and does not require an electromagnetic field as an energy source. However, an active RFID device 20 is, nonetheless, used in conjunction with the reader 30 for triggering its response. Active RFID devices generally provide improved performance (i.e., covering up to 1000 feet) by comparison to passive devices; however, active RFID devices have a finite useful life (depending upon the life of the internal battery) and are more costly than passive devices.

Hybrid RFID devices have characteristics of both passive and active devices inasmuch as such devices capture energy from a surrounding electromagnetic field but also employ a “battery assist” to improve the performance/range of communication.

Both active and passive RFID devices operate in the High Frequency (HF) and Ultra-High Frequency (UHF) radio frequency bands or between about 13.56 MHz (HF) to about 960 MHz (UHF). Generally, RFID devices operating in the HF bands are preferable for short ranges or in connection with objects having a high metal content. Those operating in the UHF bands are preferable for longer ranges and may have greater application for the detection of hazardous biological/chemical agents as described herein. That is, mail and mail trays in a mail distribution center can be widely disbursed within a facility covering several hundred acres, hence greater communication and detection ranges may be preferable for these applications. However, due to the processing flows required in mail processing, it is also possible to place the readers at “pinch points” in the flow where items pass through portals (doorways, processing stages) and can be scanned without the need for the RFID device to respond from significant distances.

RFID devices 20 useful for practicing the invention are available from Emerson & Cuming Microwave products located in Randolph, Mass., under the tradename “SMART DEVICE”, and PLITEK located in Des Plaines, Ill., under the tradename “SMART LABELS”.

RFID readers 30 for use in conjunction with RFID devices 20 are available from a variety of sources including systems from Symbol Technologies located in Holtsville, N.Y., under the Model Nos. AR400 and DC400 and SAMsys having a place of business in Ontario, Canada under the Model Nos. MP310 and MP 320.

In the most fundamental sense, the system 10 of the present invention can detect and provide an alert to facility operators that a mailpiece has been contaminated and should be further investigated to determine the cause/veracity of the issued warning/advisory. In one embodiment of the invention, and referring to FIG. 1, the RFID system 20, 30 is responsive to the contaminant sensor 18 by issuing a status signal 24 concerning whether the mailpiece 12C is contaminated. In this embodiment, the RFID device 20 changes the state of its identification or serial number from a first or “clean/uncontaminated” state to a second or “contaminated” state. As such, when using a RFID device 20 which has changed state, the reader 30 identifies the state change and issues a warning/advisory to facility operators. Optionally, an RFID device 20 (using received or internal energy source) can respond to the reader 30 and, optionally, issue an audible or visual advisory directly. For example, the RFID device 20 can change the appearance of an embedded Liquid Crystal Display (LCD) as well as transmitting state change information to a reader 30 when queried.

More specifically, the contaminant sensor 18 may be adapted to effect a switch closure or change in conductivity to cause a change in certain RFID identification numbers and switch settings. That is, the RFID device 20 may be configured with a checksum algorithm for confirming data integrity and minimizing the potential of false reporting due to read errors. More specifically, the checksum algorithm may comprise a select number of digits or numeric fields which may precede, succeed or otherwise be integrated in combination with the RFID identification number, e.g., serial number.

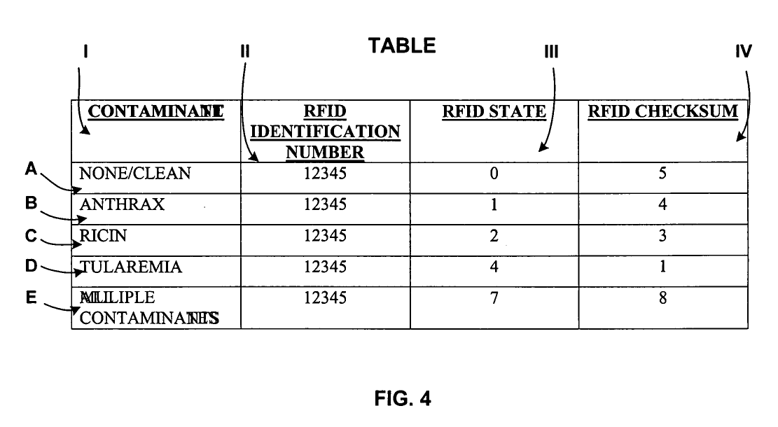

In the following example illustrated in the Table of FIG. 4, a one-digit state code and one-digit checksum follow a five-digit identification of the RFID device 20. Referring to the Table, Column I indicates whether a mailpiece contains or has been exposed to a particular contaminant, and/or to which type of contaminate. Furthermore, the identification number of the RFID device 20 is identified in Column II and each digit of the two-digit numeric suffix is identified in Columns III and IV. The first digit thereof, indicated in Column III, relates to the RFID state; and, the second digit, identified in Column IV, is associated with the RFID checksum. Moreover, the RFID device 20 is configured such that the sum of all digits, i.e., the serial number and two-digit suffix, is a multiple of a predetermined number, e.g., multiple of the number ten (10). Such checksum algorithms are commonly employed in barcode and data transmission applications to confirm data integrity. Should a digit be misread, the checksum will not match the defined algorithm (e.g., multiple of 10) signifying that the data has been corrupted in transmission. In such circumstances, the data request is to be repeated and, if the error persists, senor malfunction rather than an actual contaminant may be deemed to be the root cause of the problem. As such, the advisory/warning would be further investigated without the need for issuing an alarm.

In the clean/uncontaminated state the RFID identification number, for the purposes of illustration, is 12345 and is constant, i.e., never changes. In Row A, the RFID state is set to zero (0), and the RFID checksum is set to five (5) such that the sum of the two-digit suffix and the identification number is twenty (20). If, for example, a hazardous biological agent such as Anthrax is detected (see Row B), the RFID state changes from the numeric value of zero (0) to the numeral one (1) and the checksum digit is decremented by this value, i.e., five (5) minus one (1) to yield a numeric value of four (4). Similarly, if a different contaminant such as Ricin is detected (Row C), the RFID state changes from the numeric value of zero (0) to the numeral two (2) and the checksum digit is decremented by this value, i.e., five (5) minus two (2) to yield a numeric value of three (3). In Row D, another hazardous agent, Tularemia, effects a state change to the numeral four (4) and a checksum value of one (1). In Row E, all hazardous agents are detected to yield an RFID state of seven (7), i.e., the summation of the RFID states for the various contaminates while the checksum yields a value of eight (8) or the summation of all values in Rows B through D [(5−1)+(5−2)+(5−4)].

While the system illustrated in FIG. 1 depicts a contaminant sensor 18 which is proximal to the RFID device 20 and, essentially in wireless communication therewith, in FIG. 5, the sensor 18 and device 20 may be an integral unit, i.e., combined on a single substrate material and in direct electrical communication. Such detection device, therefore, may be employed in a broader spectrum of potential applications, i.e., not limited to detecting hazardous material on a mailpiece. Other applications may include detecting hazardous material in any area which may be a target for acts of terror, e.g., airport, luggage/luggage compartments on a plane/train, food distribution centers, etc.

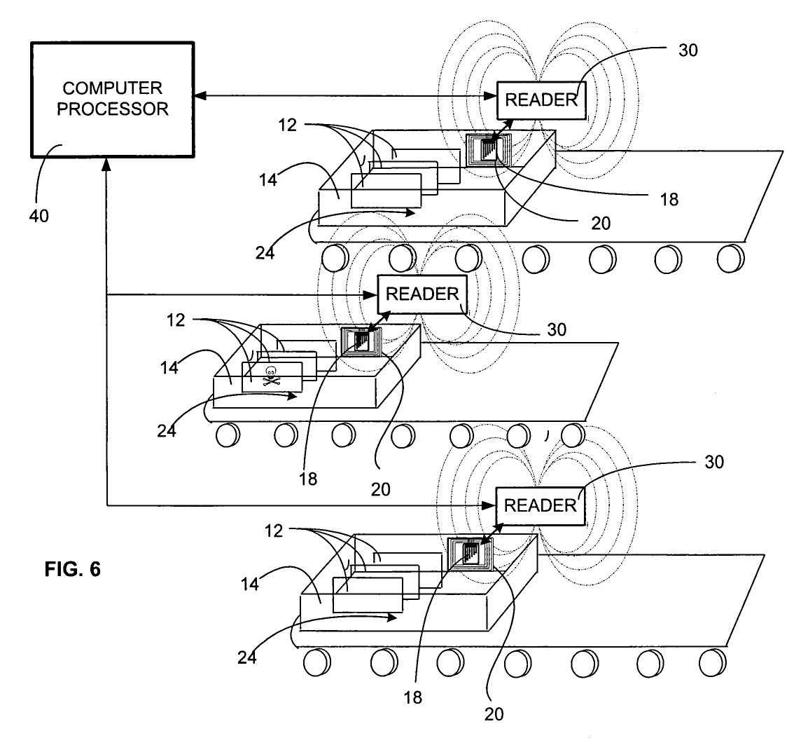

In another embodiment of the invention illustrated in FIG. 6, additional information may be compiled and processed to: (i) rapidly identify the precise location of the contaminated mailpiece, (ii) reference the location and possible cross-contamination of other mail pieces in close proximity to the contaminated mailpiece, and/or (iii) track the prior location(s) of the mailpiece to assist in determining the origin of the contaminated mailpiece.

In this embodiment, a computer processor 40 is a further component of the system for collecting data and processing information. Such system 10, therefore, may comprise multiple integral detection devices, i.e., contaminant sensors 18 and RFID devices 20, in communication with the processor 40 via the reader 30 of the RFID system 20, 30. Initially, the “clean” status of the RFID devices 20 is known by, i.e., stored, within a computer database of the processor 40. Whether active, passive or a hybrid thereof, the RFID system 20, 30 communicates with the processor 40 regarding its current or changed state to provide notification regarding whether a mailpiece 12C has been exposed to or contains a hazardous biological agent/contaminate. As discussed earlier, a state condition can be encoded into a numeric field of the RFID's serial number.

The RFID systems 20, 30 may communicate information to the computer processor 40 regarding, not only its current status, but also, its location. In this regard, the location of a reader 30 can be recorded and used by the processor 40 to track the whereabouts of the detection devices 18, 20 and associated mailpieces 12 as they enter or exit a mail distribution center. The use of multiple readers 30 within a facility can further refine the tracking information internally of the distribution center. Further, such information can be analyzed by the processor 40 to determine whether other mailpieces may have been sufficiently proximal to a contaminated mailpiece that these mailpieces must also be quarantined and/or examined before further transport and/or delivery.

Such information may be useful in determining secondary or tertiary levels of contamination by knowing where and when and how both contaminated and non-contaminated mailpieces may have entered or exited a facility as well as the pathways of such mailpieces through the facility. For example, when mailpieces 12 are first placed within trays at a particular postal facility or distribution center, an RFID device 20 attached to each tray will be “tagged” with location data (i.e., including a time/date stamp). This information may be tracked and read by readers 30 (i.e., disposed at various locations within the same postal facility or at new postal facilities) to record the pathways of mail pieces, and stored within a database of the processor 40. Alternatively, if the RFID device 20 has read/write memory capability, the location/historical information may be updated within the memory of RFID device 20.

Once tagged, the mailpieces within each tray are processed in a conventional manner. That is, the mailpieces 12 are aligned and faced in a common orientation in preparation for manual or automated processing by a Multiple Line Optical Character Reader (MLOCR) or a Remote BarCode (RBC) Reader. Those which are processed by automated equipment are sent to bar code sorters where they are grouped in accordance with the destination ZIP code obtained by reading the POSTNET barcode on the face of the respective mailpiece. Manually processed mailpieces 12 are similarly sorted depending upon the destination ZIP code.

Once a mailpiece 12C has been identified as being contaminated others which may be “suspect”, i.e., a mailpiece/tray which may have been close to or in the same general area as the contaminated mailpiece, may be identified. That is, inasmuch as the location data of each tagged RFID device 20, has been stored within the processor database, “suspect groups” may also be identified, rerouted and/or out-sorted for evaluation prior to final delivery. For example, a suspect group may include all mailpieces passing through a particular location, facility, or pathways through a facility, after a particular time. Alternatively, or additionally, a suspect group may be all mailpieces being inducted to a particular facility within a specified period of time. The mail trays containing suspect mail pieces that are known to have been in the vicinity of a contaminated mail piece can then be “tagged” by changing the data in the RFID tag on each suspect mail tray. This will enable clarity and eliminate potential confusion for future processing and investigation activities by enabling the mail trays with the RFID devices to identify themselves as either suspect on not suspect.

The system, therefore, may be adapted to track the location of suspect mailpieces/trays in a mail distribution center. When one RFID device has change status, the reader and processor (capable of scanning and analyzing the stored mailpiece identifying data), can issue an alert signal to all other RFID devices (i.e., those having read/write capability) that are currently or have been previously in close proximity to the contaminated RFID device, mailpiece or mail tray. The reader 30 can then effect a change of state condition in multiple RFID devices such that mailpieces or mail trays which are suspect can be quarantined for further study or investigation. As such, historical interactions of the mailpieces or containers may be tracked. Commonly-owned U.S. Pat. No. 6,770,831 discusses a method and system for re-routing mail in a mail distribution system and contains additional detail concerning the way that “suspect mailpieces” may be defined and how such information may be stored. Accordingly, the information contained in the specification thereof is hereby incorporated by reference in its entirety.

The signal strength issued by an RFID device 20 can be monitored by several readers (or other device/receiver for measuring signal strength) spatially located or arranged to compare differences and, via directional antennas or mathematical manipulation such as triangulation, can determine the precise location of the RFID device 20. Of course, other more sophisticated means, such as the use of a Differential Global Positioning System (DGPS), may also be readily adapted for this purpose. An RFID reader 30 may periodically interrogate the devices 20 to determine whether the state condition has changed. As mentioned earlier, information relative to the location of the reader 30 may be recorded to enable the system to track, record and identify mailpieces for possible cross contamination or to locate the origin of a contaminated mailpiece 12.

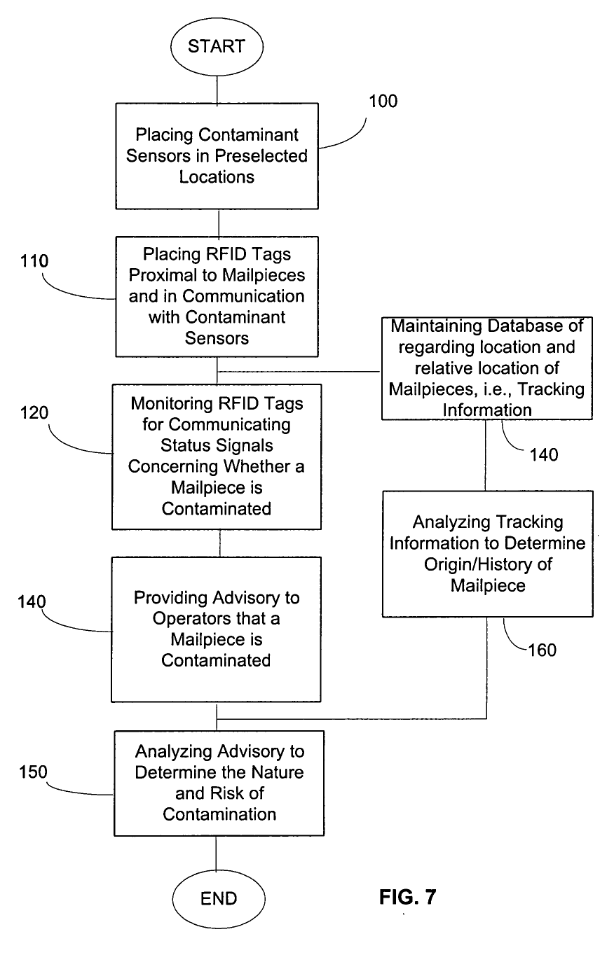

In FIG. 7, the method steps employed for practicing the invention are shown in flow diagram format. In the description of the method, it will be assumed that multiple sensors 18 and devices 20 are employed for use in one or several large mail distribution centers. In step 100, contaminant sensors 18 are disposed in predefined locations corresponding to areas of interest or, at minimum, areas where mailpieces 12 will pass as they are transported within the mail distribution center. In step 110, RFID devices 20 are disposed proximal to and travel with the mailpieces 12. While RFID devices 20 may be placed in combination with individual mailpieces 12, a more pragmatic implementation of the invention includes the step of affixing the RFID devices 20 directly to the mail-carrying trays 14 which, in terms of total numbers, are far fewer than the number of total mailpieces being handled and/or transported within a mail distribution center. In step 120, the status signals issued by the RFID systems 20, 30 are monitored for communicating when a particular mailpiece is contaminated. The execution of this step 120 occurs periodically or in real time.

The contaminant sensor(s) 18 will first send an alert signal to the RFID device(s) upon sensing a contaminant, and the RFID system 20, 30, in response to the alert signal, will issue a status signal indicative of a change in state, i.e., from clean to contaminated. Upon sensing or detecting a change in state, in step 130 an advisory notice is sent to facility operators that a mailpiece has been contaminated and may require immediate attention or further investigation. Additionally, in step 140, the method may include the maintenance of a computer database for storing information relative to the location of various mailpieces and/or the relative location thereof. This information can be critical for determining the origin of the contaminated mailpiece and/or the secondary or tertiary effects caused by cross-contamination of mailpieces. Subsequent steps such as analyzing the severity of the advisory in step 150 (i.e., to determine the nature and health risks caused by the contamination), and analyzing the tracking information in step 160 (i.e., to determine the possible point of origin and risks associated with cross-contamination), may be performed manually or with the assistance of computer processing equipment.

While the status signal issued by the RFID system 20, 30 has been described in the context of changing the state of an identification code or the issuance of an audible signal, it will be appreciated that the status signal 24 may take other forms and/or manifestations. For example, the status signal 24 may be communicated by a lighted diode on the face of the RFID device 20. Light from the diode may be visually detected by a facility operator or detected by an electronic scanning device positioned to intercept the emitted light. As mentioned earlier, a liquid crystal display may also be used and is advantageous in low power applications, i.e., able to produce a highly visible cue with low power consumption. In the broadest sense of the invention, therefore, the status signal is any means for cuing facility operators, (audible, visual, radio, etc.) whether the operators are proximal or remote from the RFID device when a change of status has been identified.

Although the invention has been described with respect to a preferred embodiment thereof, it will be understood by those skilled in the art that the foregoing and various other changes, omissions and deviations in the form and detail thereof may be made without departing from the scope of this invention.

What is claimed is:

1. A system for detecting a mailpiece contaminated by hazardous agents/materials comprising:
a contaminant sensor disposed for detecting the presence of a hazardous agent/material, the contaminant sensor issuing an alert signal indicating that a hazardous agent/material has been sensed;
an Radio Frequency IDentification (RFID) system in communication with the contaminant sensor; and
the RFID system responsive to the alert signal for issuing a change of status signal when a mailpiece is contaminated.

2. The system according to claim 1 wherein the mailpiece is disposed in a mailpiece carrying tray, and wherein the RFID system includes an RFID device connected to the tray.

3. The system according to claim 1 wherein the RFID system includes an RFID device coupled to the mailpiece, and the contaminant sensor is attached to a mailpiece carrying tray.

4. The system according to claim 1 wherein the contaminant sensor is coupled to the RFID device.

5. The system according to claim 1 wherein the RFID system includes an RFID device which is adapted to change a state condition in response to the alert signal.

6. The system according to claim 1 wherein the RFID system includes a plurality of RFID devices and further comprising a processor in communication with the plurality of RFID devices, the processor for maintaining a database of tracking information related to each of the plurality of RFID devices.

7. The system according to claim 6 wherein the tracking information includes proximity information.

8. The system according to claim 6 wherein the processor is adapted to issue an alert signal to RFID devices that have been in close proximity to a contaminated RFID device.

9. The system according to claim 5 wherein the RFID device includes a checksum algorithm for maintaining data integrity.

10. A detection device for identifying hazardous agents/materials comprising:
a contaminant sensor for detecting the presence of a hazardous agent/material, the contaminant sensor issuing an alert signal indicating that a hazardous agent/material has been sensed; and
a Radio Frequency Identification (RFID) device coupled to the contaminant sensor;
wherein the RFID device is responsive to the alert signal for changing a state condition of the RFID device.

11. The detection device according to claim 10 wherein the RFID device is an active device.

12. The detection device according to claim 10 wherein the RFID device is a passive device.

13. The detection device according to claim 10 wherein the RFID device includes a state value stored therein that is capable of being changed and which is indicative of the state condition.

14. The system according to claim 13 wherein the RFID device further includes a checksum algorithm and an identification number stored therein, the identification number, state value and checksum algorithm used by the RFID device to create a checksum value.

15. The system according to claim 1 wherein the contaminant sensor is one of a microelectromechanical, magnetoelastic, microphysiometer, nanowire, waveguide, liquid crystal, immunoassay and distributed dust sensor.

16. A method for detecting a mailpiece contaminated by hazardous agents/materials comprising:
disposing a contaminant sensor proximal to the mailpiece for detecting the presence of a hazardous agent/material at the mailpiece, the contaminant sensor issuing an alert signal indicating that a hazardous agent/material has been sensed;
and
providing a Radio Frequency Identification (RFID) system having an RFID device proximal to the mailpiece and in communication with the contaminant sensor, the RFID device changing a state in response to the alert signal for providing notification that the mailpiece is contaminated.

17. The method according to claim 16 wherein the mailpiece is disposed in a mailpiece carrying tray, and further comprising the step of attaching the RFID device to the mailpiece carrying tray.

18. The method according to claim 16 further comprising the step of attaching the contaminant sensor to the RFID device.

19. The method according to claim 16 further comprising the step of providing a plurality of RFID devices and a processor in communication with the plurality of RFID devices, and wherein the processor maintains a database of tracking information related to each of the plurality of RFID devices.

20. The method according to claim 19 wherein the processor records proximity information related to the plurality of RFID devices, and wherein the processor is adapted to issue an alert signal to the plurality of RFID devices that have been in close proximity to a contaminated RFID device.
